(19) United States
(12) Patent Application Publication
(10) Pub. No.: US 2026/0105637 A1
(43) Pub. Date: Apr. 16, 2026

(54) METHOD AND SYSTEM FOR CALIBRATING CAMERAS

(71) Applicant: Auris Health, Inc., Santa Clara, CA (US)

(72) Inventors: Juri PLATONOV, Munich (DE); Yiming XU, Sunnyvale, CA (US); Bernhard Adolf FUERST, Sunnyvale, CA (US)

(21) Appl. No.: 19/420,507

(22) Filed: Dec. 15, 2025

Related U.S. Application Data
(62) Division of application No. 18/152,606, filed on Jan. 10, 2023.

(51) Int. Cl. G06T 7/80; A61B 34/20; A61B 90/00; G06T 7/246; G06T 7/73; H04N 17/00; H04N 23/90

(52) U.S. Cl. CPC G06T 7/80; A61B 90/361; G06T 7/246; G06T 7/73; H04N 17/002; H04N 23/90; A61B 2034/2057; A61B 2090/371; A61B 2090/3937; G06T 2207/10016; G06T 2207/10068; G06T 2207/30004; G06T 2207/30204; G06T 2207/30244

(57) ABSTRACT

A method performed by a surgical system that includes cameras within an operating room. The system receives a first image from a first camera, representing a first FOV that has an object at a first location. The system receives a second image from a tracking camera having the object at the first location, and determines a first pose of the first camera based on the first and second images. The system receives a third image captured by a second camera, representing a second, different FOV, and having the object at a second location. The system receives a fourth image captured by the tracking camera having the object at the second location, and determines a second pose of the second camera based on the third and fourth images. The system determines a relative spatial transformation between the first and second cameras based on the first and second poses.

### CROSS-REFERENCE TO RELATED APPLICATION

This patent application is a divisional of U.S. patent application Ser. No. 18/152,606, filed Jan. 10, 2023, which is incorporated herein by reference in its entirety.

### FIELD

Various embodiments of the disclosure relate generally to surgical systems, and more specifically to a surgical system that calibrates one or more cameras.

### BACKGROUND

Minimally-invasive surgery, MIS, such as laparoscopic surgery, uses techniques that are intended to reduce tissue damage during a surgical procedure.

Laparoscopic procedures typically call for creating a number of small incisions in the patient, e.g., in the abdomen, through which several surgical tools such as an endoscope, a blade, a grasper, and a needle, are then inserted into the patient. A gas is injected into the abdomen which insufflates the abdomen thereby providing more space around the tips of the tools, making it easier for the surgeon to see (via the endoscope) and manipulate tissue at the surgical site. MIS can be performed faster and with less surgeon fatigue using a surgical robotic system in which the surgical tools are operatively attached to the distal ends of robotic arms, and a control system actuates the arm and its attached tool. The tip of the tool will mimic the position and orientation movements of a handheld user input device (UID) as the latter is being manipulated by the surgeon. The surgical robotic system may have multiple surgical arms, one or more of which has an attached endoscope and others have attached surgical instruments for performing certain surgical actions.

Control inputs from a user (e.g., surgeon or other operator) are captured via one or more user input devices and then translated into control of the robotic system. For example, in response to user commands, a tool drive having one or more motors may actuate one or more degrees of freedom of a surgical tool when the surgical tool is positioned at the surgical site in the patient.

### SUMMARY

An increasing number of applications may benefit from the usage of images, e.g., video that are captured by multiple image sensors, such as video cameras and depth cameras. Examples of such applications may include telepresence, data collection for various computer training purposes (e.g., training a motion controller of an autonomous vehicle), and event detection during a surgical procedure in an operating room. For example, multiple video cameras may be used inside hospital facilities, such as an operating room in order to monitor events and surgical workflow during a surgical procedure (e.g., monitoring the surgical tasks performed by a surgeon). Cameras within such a setting may be used to monitor activities, detect events of interest, and analyze efficiency for training and improvement purposes. For example, in addition to detecting a person or an object entering a room or space that is being observed by a camera, the system may be able to track the movement of objects through multiple cameras, such as gurneys moving from room to room. As a result of maximizing the observable range of such large rooms, some cameras may not have overlapping fields of view (FOV), where one camera may be directed towards a door, while another camera may be directed in a direction opposite of the door.

When collecting video data from multiple cameras, it may be desirable to correlate their spatial information (e.g., position and orientation in space with respect to one another) to better enable analysis of the video data to perform operations, such as detecting activities or events. For example, when detecting movement of an object, such as a patient's bed or gurney, within an operating room, the object may move out of one camera's FOV and into another camera's FOV. Spatial information indicating the relationship between the two cameras is then used in order to maintain consistent tracking of the object as the object moves between FOVs. This provides meaningful deductions and analysis of detected events.

In order to correlate spatial information, cameras may be spatial calibrated by calculating intrinsic and extrinsic parameters (or properties) of the camera. Intrinsic parameters may include properties associated with a particular camera, such as physical attributes of the camera. Examples of intrinsic properties may include a focal length of (e.g., a lens of) the camera, a principal point (or optical center) of the (e.g. lens of) the camera, a skew of the camera, and a distortion of the lens of the camera. These intrinsic properties may represent a transformation from a two-dimensional (2D) image coordinate system (e.g., a pixel coordinate in a 2D captured image) to a three-dimensional (3D) coordinate system of the camera's 3D space (with respect to the camera as the origin). The extrinsic parameters may represent (or include) a transformation (e.g., position and/or orientation) from the camera's 3D coordinate to a global (or world) coordinate system. In particular, the extrinsic parameters may represent a relative spatial transformation (or pose) between two cameras. The process of estimating a camera's pose with respect to another camera may be performed through a spatial extrinsic calibration. Conventional calibration methods require that multiple cameras have at least partial overlapping FOV, where this overlapping portion includes a common reference point. These methods, however, may be unable to accurately calibrate cameras with non-overlapping fields of view, and especially for such cameras that are a part of static (e.g., not movable) image sensor setups. Thus, there is a need for a surgical system that is configured to calibrate (e.g., static) image sensors with both overlapping and non-overlapping FOV.

The present disclose provides a surgical system that includes a first camera, a second camera, and a tracking camera that are located within one or more operating rooms, and that is configured to perform spatial calibration between the cameras. For example, the two cameras may be situated within a room (e.g., mounted on separate walls), and may be used for event detection within the operating room. In particular, the system receives a first image captured by the first camera, the first image representing a first FOV of the first camera that has an object, such as a calibration pattern, at a first location in the operating room. The system receives a second image captured by the tracking camera, the second image having the object at the first location, and determines a first pose of the first camera based on the first image and the second image. Specifically, the pose of the first camera may be with respect to the tracking camera such that a position and/or an orientation of the first camera is within a coordinate system of the tracking camera. The system receives a third image captured by the second camera, where the third image represents a second FOV of the second camera that does not overlap with the first FOV, and has the object at a second location in the operating room. The system receives a fourth image captured by the tracking camera, where the fourth image has the object at the second location, and determines a second pose of the second camera based on the third image and the fourth image. Thus, both poses of the first and second cameras are within the coordinate system of the tracking camera. The system determines a relative spatial transformation, e.g., a pose, between the first camera and the second camera based on the first and second poses. As a result, the system is able to know the relative spatial information between the two cameras, which allows the system to effectively track movement through all observations between the cameras even when the cameras are set up so that they cover different room locations and do not have overlapping FOV.

In one embodiment, the tracking camera comprises a third FOV that includes both of the first and second locations. For example, the tracking camera may be positioned on an operating rooms ceiling, and include a wide-angled lens in order to see the inside of the operating room. In one embodiment, the tracking camera is stationary while the object is moved from the first location to the second location. In another embodiment, the third image and the fourth image are captured by the second camera and the tracking camera, respectively, before the first image and the second image are captured by the first camera and the tracking camera, respectively. In one embodiment, the first and second images are captured simultaneously by the first camera and the tracking camera, respectively, and the third and second images are captured simultaneously by the third camera and the tracking camera, respectively.

In one embodiment, the object remains stationary at the first location as the first image and second image are captured by the first camera and tracking camera, respectively, and the object remains stationary at the second location as the third image and the fourth image are captured by the third camera and the tracking camera, respectively. In another embodiment, the object is not attached to any of the first camera, the second camera, and the tracking camera.

In one embodiment, determining the first pose of the first camera based on the first image and the second image includes determining a third pose of the first camera with respect to the object at the first location using the first image; and determining a fourth pose of the tracking camera with respect to the object at the first location using the second image. In another embodiment, the relative spatial transformation indicates a position and an orientation of the second camera with respect to the first camera. In one embodiment, the object comprises a calibration pattern that is moved from the first location to the second location by a user within the operating room, while the first camera, the second camera, and the tracking camera remain in place.

The present disclosure provides a surgical system that includes the first camera, the second camera, and a (e.g., tracking) marker within an operating room for calibrating the two cameras. In particular, the system receives a first image captured by the first camera, the first image representing a first FOV of the first camera including the marker at a first location in the operating room, and determines a first pose of the first camera based on the first image and the first location of the marker. The system receives a second image captured by the second camera, the second image representing a second FOV of the second camera that does not overlap with the first FOV and includes the marker at a second location in the operating room, and determines a second pose of the second camera based on the second image and the second location of the marker. The system determines a relative spatial transformation between the first camera and the second camera based on the first and second poses, where the first camera and the second camera remain stationary as the marker is moved from the first location to the second location.

In one embodiment, the marker is fixedly coupled to an object that includes a calibration pattern, where the object is at the first location captured in the first image and the second image. The system determines, using a tracking device, a third location at which the marker is fixedly coupled to the calibration object, where determining the first pose of the first camera includes determining a third pose of the first camera with respect to the calibration pattern while the object is at the first location; and determining a fourth pose of the tracking device with respect to the calibration pattern using the third location of the marker. In another embodiment, determining the fourth pose of the tracking device includes determining a fifth pose of the tracking device with respect to the marker according to the third location of the marker; retrieving a sixth pose of the marker with respect to the calibration pattern (e.g., from memory of the surgical system); and adjusting the fifth pose according to the sixth pose. In one embodiment, the marker and the calibration pattern is one integrated unit. In another embodiment, the tracking device is an infrared sensor, and the marker is an infrared tag. In another embodiment, the tracking device is a camera, and the marker is a visible pattern.

The present disclosure provides a surgical system that includes the first camera and the second camera within the operating room, where the system receives a first image captured by the first camera, the first image representing a first FOV that includes an object at a first location in the operating room, and determines a first pose of the first camera based on the first image and a tracking marker within the operating room, such as being disposed on the ceiling of the operating room. The system receives a second image captured by the second camera, the second image representing a second FOV of the second camera that does not overlap with the first FOV, and has the object at a second location in the operating room, and determines a second pose of the second camera based on the second image and the tracking marker. The system determines the relative spatial transformation between the first and second cameras based on the first and second poses.

In one embodiment, object includes a tracking device. The method detects, using the tracking device, the tracking marker while the object is at the first location, the first pose is determined based on the detection of the tracking marker while the object is at the first location; and detects using the tracking device, the tracking marker while the object is at the second location, where the second pose is determined based on the detection of the tracking marker while the object is at the second location.

In one embodiment, the method includes tracking, using the tracking device, movement of the object from the first location to the second location, where the second pose is determined based on the tracked movement of the object. In another embodiment, the tracking device and the object are one integrated unit. In another embodiment, the system further determines a third pose of the tracking marker with respect to the object based on the detection of the tracking marker while the object is at the first location; and determines a fourth pose of the first camera with respect to the object based on the first image, where the first pose of the first camera is based on the third pose and the fourth pose. In one embodiment, determining the third pose of the tracking marker includes determining a fifth pose of the tracking marker with respect to the tracking device based on the detection of the tacking marker; receiving a sixth pose of the tracking device with respect to the object; and adjusting the fifth pose according to the sixth pose.

In one embodiment, the tracking device is a tracking camera, where detecting the tracking marker while the object is at the first location includes receiving a third image captured by the tracking camera, the third image representing a third FOV of the tracking camera and including the tracking marker. In one embodiment, the tracking marker is an infrared (IR) tag, and the tracking device is an IR sensor. In another embodiment, the tracking marker is a radio frequency (RF) tag, and the tracking device is a RF sensor. In one embodiment, the object includes a calibration pattern that is moved from the first location to the second location by a user or an autonomous robot within the operating room, while the first camera and the second camera remain in place.

The above summary does not include an exhaustive list of all embodiments of the disclosure. It is contemplated that the disclosure includes all systems and methods that can be practiced from all suitable combinations of the various embodiments summarized above, as well as those disclosed in the Detailed Description below and particularly pointed out in the claims. Such combinations may have particular advantages not specifically recited in the above summary.

### BRIEF DESCRIPTION OF THE DRAWINGS

The embodiments are illustrated by way of example and not by way of limitation in the figures of the accompanying drawings in which like references indicate similar elements. It should be noted that references to “an” or “one” embodiment of this disclosure are not necessarily to the same embodiment, and they mean at least one. Also, in the interest of conciseness and reducing the total number of figures, a given figure may be used to illustrate the features of more than one embodiment, and not all elements in the figure may be required for a given embodiment.

FIG. 1 shows a pictorial view of an example surgical system in an operating arena.

FIG. 2 is a block diagram of the surgical system that calibrates one or more cameras according to one embodiment.

FIG. 3 is a flowchart of a process for an embodiment of calibrating one or more cameras using a tracking device.

FIG. 4 illustrate several stages that show calibrating two cameras using a tracking device.

FIG. 5 is a flowchart of a process for an embodiment of calibrating one or more cameras using a tracking device and a tracking marker.

FIG. 6 illustrate several stages that show calibrating two cameras using a tracking device and a tracking marker.

FIG. 7 is a flowchart of a process for an embodiment of calibrating one or more cameras using a tracking device and a tracking marker.

FIG. 8 illustrate several stages that show calibrating two cameras using a tracking device and a tracking marker.

### DETAILED DESCRIPTION

Several embodiments of the disclosure with reference to the appended drawings are now explained. Whenever the shapes, relative positions and other embodiments of the parts described in a given embodiment are not explicitly defined, the scope of the disclosure here is not limited only to the parts shown, which are meant merely for the purpose of illustration. Also, while numerous details are set forth, it is understood that some embodiments may be practiced without these details. In other instances, well-known circuits, structures, and techniques have not been shown in detail so as not to obscure the understanding of this description. Furthermore, unless the meaning is clearly to the contrary, all ranges set forth herein are deemed to be inclusive of each range's endpoints.

In one embodiment, a “pose” may refer to the relative position and/or orientation of one object or reference frame with respect to another object or reference frame. In particular, a pose may be a six-degrees-of-freedom (6DOF) variable that indicates a three-dimensional (3D) location and a 3D orientation of an object relative to an object in 3D space or a reference frame. For instance, the location may include three parameters, such as a X-coordinate, a Y-coordinate, and a Z-coordinate for a 3D Cartesian coordinate system, and the orientation may include three parameters, such as a set of three rotation angles about three axes of the coordinate system (e.g., yaw, pitch, and roll of the Euler angles). In one embodiment, a pose may be a relative spatial transformation that may include a transformation matrix, P, which may include a translation vector that indicates the position of the object with respect to an origin of a reference frame in space and/or a rotational matrix that indicates the orientation of the object with respect to the reference frame.

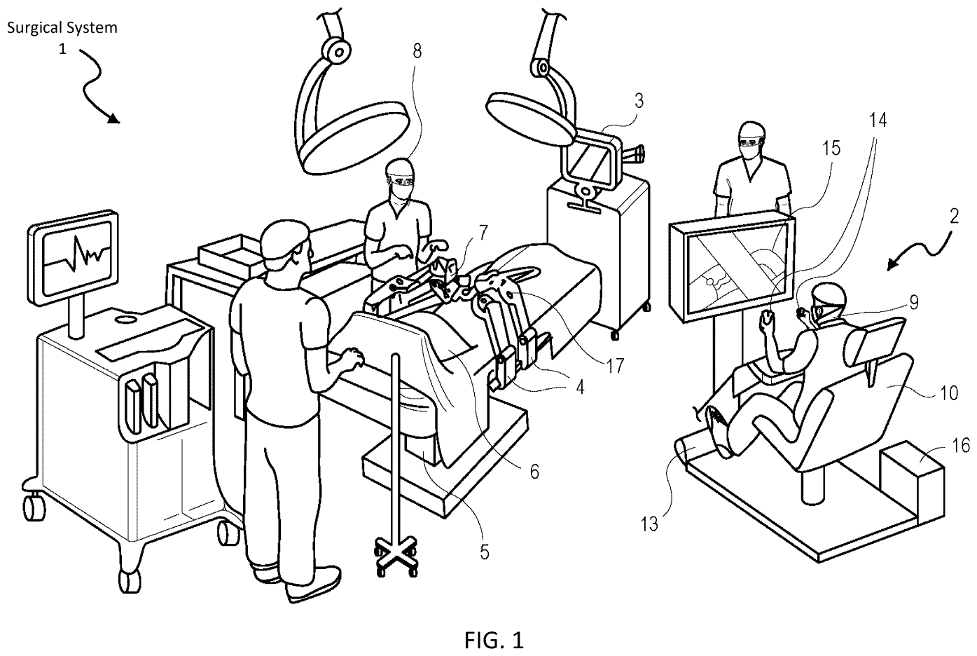

FIG. 1 shows a pictorial view of an example (e.g., laparoscopic) surgical system (which hereafter may be referred to as “system”) 1 in an operating arena (or room). The system 1 includes a user console 2, a control tower 3, and one or more surgical robotic arms 4 at a surgical robotic table (surgical table or surgical platform) 5. In one embodiment, the arms 4 may be mounted to a table or bed on which the patient rests as shown in the example of FIG. 1. In one embodiment, at least some of the arms 4 may be configured differently. For example, at least some of the arms may be mounted on a ceiling, sidewall, or in another suitable structural support, such as a cart separate from the table. The system 1 can incorporate any number of devices, tools, or accessories used to perform surgery on a patient 6. For example, the system 1 may include one or more surgical tools (instruments) 7 used to perform surgery (surgical procedure). A surgical tool 7 may be an end effector that is attached to a distal end of a surgical arm 4, for executing a surgical procedure.

Each surgical tool 7 may be manipulated manually, robotically, or both, during the surgery. For example, the surgical tool 7 may be a tool used to enter, view, perform a surgical task, and/or manipulate an internal anatomy of the patient 6. In an embodiment, the surgical tool 7 is a grasper that can grasp tissue of the patient. The surgical tool 7 may be controlled manually, by a bedside operator 8; or it may be controlled robotically, via actuated movement of the surgical robotic arm 4 to which it is attached. For example, when manually controlled an operator may (e.g., physically) hold a portion of the tool (e.g., a handle), and may manually control the tool by moving the handle and/or pressing one or more input controls (e.g., buttons) on the (e.g., handle of the) tool. In another embodiment, when controlled robotically, the surgical system may manipulate the surgical tool based user input (e.g., received via the user console 2, as described herein).

Generally, a remote operator 9, such as a surgeon or other operator, may use the user console 2 to remotely manipulate the arms 4 and/or the attached surgical tools 7, e.g., during a teleoperation. The user console 2 may be located in the same operating room as the rest of the system 1, as shown in FIG. 1. In other environments however, the user console 2 may be located in an adjacent or nearby room, or it may be at a remote location, e.g., in a different building, city, or country. The user console 2 may include one or more components, such as a seat 10, one or more foot-operated controls (or foot pedals) 13, one or more (handheld) user-input devices (UIDs) 14, and at least one display 15. The display is configured to display, for example, a view of the surgical site inside the patient 6. The display may be configured to display image data (e.g., still images and/or video). In one embodiment, the display may be any type of display, such as a liquid crystal display (LCD), a light-emitting diode (LED) display, an organic LED (OLED) display, a head-mounted display (HMD), etc. In some embodiments, the display may be a 3D immersive display that is for displaying 3D (surgical) presentations. For instance, during a surgical procedure one or more endoscopes (e.g., endoscopic cameras) may be capturing image data of a surgical site, which the display presents to the user in 3D. In one embodiment, the 3D display may be an autostereoscopic display that provides 3D perception to the user without the need for special glasses. As another example, the 3D display may be a stereoscopic display that provides 3D perception with the use of glasses (e.g., via active shutter or polarized).

In another embodiment, the display 15 may be configured to display at last one graphical user interface (GUI) that may provide informative and/or interactive content, to thereby assist a user in performing a surgical procedure with one or more instruments in the surgical system 1. For example, some of the content displayed may include image data captured by one or more endoscopic cameras, as described herein. In another embodiment, the GUI may include selectable UI items, which when manipulated by the user may cause the system to perform one or more operations. For instance, the GUI may include a UI item as interactive content to switch control between robotic arms. In one embodiment, to interact with the GUI, the system may include input devices, such as a keyboard, a mouse, etc. In another embodiment, the user may interact with the GUI using the UID 14. For instance, the user may manipulate the UID to navigate through the GUI, (e.g., with a cursor), and to make a selection may hover the cursor over a UI item and manipulate the UID (e.g., selecting a control or button). In some embodiments, the display may be a touch-sensitive display screen. In this case, the user may perform a selection by navigating and selecting through touching the display. In some embodiments, any method may be used to navigate and/or select a UI item.

As shown, the remote operator 9 is sitting in the seat 10 and viewing the user display 15 while manipulating a foot-operated control 13 and a handheld UID 14 in order to remotely control one or more of the arms 4 and the surgical tools 7 (that are mounted on the distal ends of the arms 4.) In some embodiments, the bedside operator 8 may also operate the system 1 in an “over the bed” mode, in which the beside operator (user) is now at a side of the patient 6 and is simultaneously manipulating a robotically-driven tool (end effector as attached to the arm 4), e.g., with a handheld UID 14 held in one hand, and a manual laparoscopic tool. For example, the bedside operator's left hand may be manipulating the handheld UID to control a robotic component, while the bedside operator's right hand may be manipulating a manual laparoscopic tool. Thus, in these variations, the bedside operator may perform both robotic-assisted minimally invasive surgery and manual laparoscopic surgery on the patient 6.

During an example procedure (surgery), the patient 6 is prepped and draped in a sterile fashion to achieve anesthesia. Initial access to the surgical site may be performed manually while the arms of the system 1 are in a stowed configuration or withdrawn configuration (to facilitate access to the surgical site.) Once access is completed, initial positioning or preparation of the system 1 including its arms 4 may be performed. Next, the surgery proceeds with the remote operator 9 at the user console 2 utilizing the foot-operated controls 13 and the UIDs 14 to manipulate the various end effectors and perhaps an imaging system, to perform the surgery. Manual assistance may also be provided at the procedure bed or table, by sterile-gowned bedside personnel, e.g., the bedside operator 8 who may perform tasks such as retracting tissues, performing manual repositioning, and tool exchange upon one or more of the robotic arms 4. Non-sterile personnel may also be present to assist the remote operator 9 at the user console 2. When the procedure or surgery is completed, the system 1 and the user console 2 may be configured or set in a state to facilitate post-operative procedures such as cleaning or sterilization and healthcare record entry or printout via the user console 2.

In one embodiment, the remote operator 9 holds and moves the UID 14 to provide an input command to drive (move) one or more robotic arm actuators 17 (or driving mechanism) in the system 1 for teleoperation. The UID 14 may be communicatively coupled to the rest of the system 1, e.g., via a console computer system 16 (or host). The UID 14 can generate spatial state signals corresponding to movement of the UID 14, e.g., position and orientation of the handheld housing of the UID, and the spatial state signals may be input signals to control motions of the robotic arm actuators 17. The system 1 may use control signals derived from the spatial state signals, to control proportional motion of the actuators 17. In one embodiment, a console processor of the console computer system 16 receives the spatial state signals and generates the corresponding control signals. Based on these control signals, which control how the actuators 17 are energized to drive a segment or link of the arm 4, the movement of a corresponding surgical tool that is attached to the arm may mimic the movement of the UID 14. Similarly, interaction between the remote operator 9 and the UID 14 can generate for example a grip control signal that causes a jaw of a grasper of the surgical tool 7 to close and grip the tissue of patient 6.

The system 1 may include several UIDs 14, where respective control signals are generated for each UID that control the actuators and the surgical tool (end effector) of a respective arm 4. For example, the remote operator 9 may move a first UID 14 to control the motion of an actuator 17 that is in a left robotic arm, where the actuator responds by moving linkages, gears, etc., in that arm 4. Similarly, movement of a second UID 14 by the remote operator 9 controls the motion of another actuator 17, which in turn drives other linkages, gears, etc., of the system 1. The system 1 may include a right arm 4 that is secured to the bed or table to the right side of the patient, and a left arm 4 that is at the left side of the patient. An actuator 17 may include one or more motors that are controlled so that they drive the rotation of a joint of the arm 4, to for example change, relative to the patient, an orientation of an endoscope or a grasper of the surgical tool 7 that is attached to that arm. Motion of several actuators 17 in the same arm 4 can be controlled by the spatial state signals generated from a particular UID 14. The UIDs 14 can also control motion of respective surgical tool graspers. For example, each UID 14 can generate a respective grip signal to control motion of an actuator, e.g., a linear actuator that opens or closes jaws of the grasper at a distal end of surgical tool 7 to grip tissue within patient 6.

In some embodiments, the communication between the surgical robotic table 5 and the user console 2 may be through a control tower 3, which may translate user commands that are received from the user console 2 (and more particularly from the console computer system 16) into robotic control commands that transmitted to the arms 4 on the surgical table 5. The control tower 3 may also transmit status and feedback from the surgical table 5 back to the user console 2. The communication connections between the surgical table 5, the user console 2, and the control tower 3 may be via wired (e.g., optical fiber) and/or wireless links, using any suitable one of a variety of wireless data communication protocols, such as BLUETOOTH protocol. Any wired connections may be optionally built into the floor and/or walls or ceiling of the operating room. The system 1 may provide video output to one or more displays, including displays within the operating room as well as remote displays that are accessible via the Internet or other networks. The video output or feed may also be encrypted to ensure privacy and all or portions of the video output may be saved to a server or electronic healthcare record system.

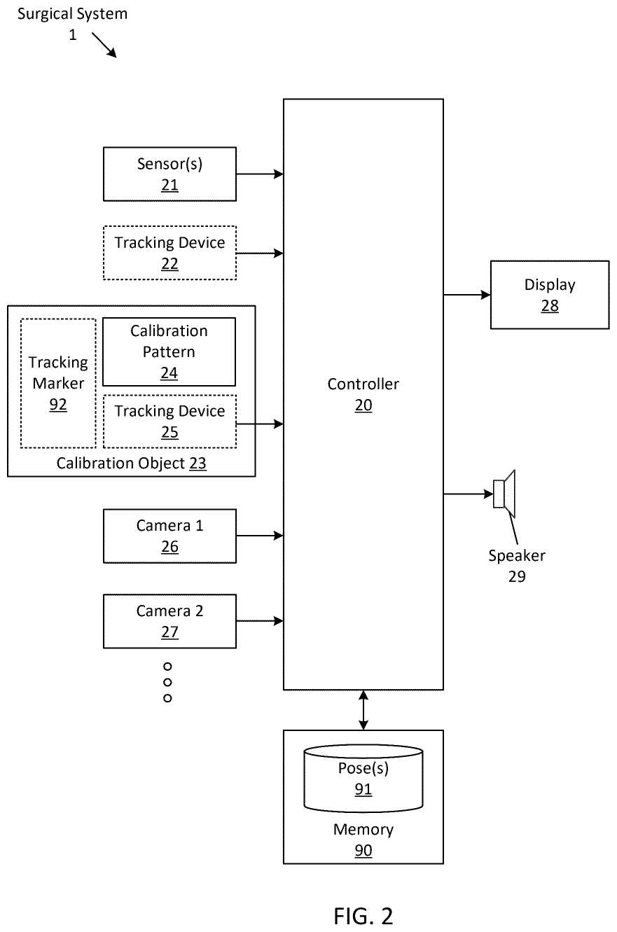

FIG. 2 is a block diagram of the surgical system 1 that calibrates one or more cameras according to one embodiment. The system includes one or more (e.g., electronic) components or elements, such as a controller 20, a sensor 21, a tracking device 22, a calibration object 23, a first camera 26, a second camera 27, a display 28, a speaker 29, and memory 90. In one embodiment, the system may include more or fewer elements, such as having one or more (different) sensors and/or not including the calibration object, the speaker, or the display. In another embodiment, at least some of the elements of the system may be optional, such as the tracking device 22, the tracking marker 92 and/or the tracking device 25. In one embodiment, the system may include one or more other elements not shown, such as having one or more robotic arms 4, as shown in FIG. 1.

In some embodiments, at least some of the elements may be a part of a single electronic device. For example, the controller 20 and the memory 90 may be housed within the control tower 3 shown in FIG. 1. In another embodiment, at least some of the elements may be separate or a part of separate electronic devices with respect to one another. For instance, the tracking device 22 may be a separate electronic device that is positioned, e.g., mounted, within an operating room in which at least a part of the surgical system 1 is located.

In one embodiment, the elements of the surgical system may be communicatively coupled with the controller 20 and/or each other in order to exchange digital data. For example, the controller may be configured to wirelessly communicate, via a network and through a wireless connection, with one or more elements, such as tracking device 22. In one embodiment, devices may communicate via any (computer) network, such as a wide area network (WAN), e.g., the Internet, a local area network (LAN), etc., through which the devices may exchange data between one another and/or may exchange data with one or more other electronic devices, such as a remote electronic server. In another embodiment, the network may be a wireless network such as a wirelessly local area network (WLAN), a cellular network, etc., in order to exchange digital data. With respect to the cellular network, the controller (e.g., via a network interface) may be configured to establish a wireless (e.g., cellular) call, in which the cellular network may include one or more cell towers, which may be part of a communication network (e.g., a 4G Long Term Evolution (LTE) network) that supports data transmission (and/or voice calls) for electronic devices, such as mobile devices (e.g., smartphones). In another embodiment, the devices may be configured to wirelessly exchange data via other networks, such as a Wireless Personal Area Network (WPAN) connection. For instance, the controller 20 may be configured to establish a wireless communication link (connection) with an element (e.g., an electronic device that includes the sensor 21) via a wireless communication protocol (e.g., BLUETOOTH protocol or any other wireless communication protocol). During the established wireless connection, the electronic device may transmit data, such as sensor data as data packets (e.g., Internet Protocol (IP) packets) to the controller 20.

In another embodiment, the controller 20 may communicatively couple with one or more electronic devices, via other methods. In particular, the controller may couple to one or more devices via a wired connection. For example, the controller 20 may couple to the cameras 26 and 27 via a High-Definition Multimedia Interface (HDMI) connection to receive image data, as a video stream that includes a series of one or more still images captured by the cameras, and may couple to the display 28 (e.g., another HDMI connection) in order to provide one or more video streams to the display to be displayed. In one embodiment, other video connections are possible, such as Digital Video Interface (DVI), Serial Digital Interface (SDI) connectors, composite video connectors, etc.

Each of the first camera 26 and the second camera 27 (e.g., a complementary metal-oxide-semiconductor (CMOS image sensor) may be an electronic device that is configured to capture video (and/or image) data (e.g., as a series of still images). In particular, each of the cameras is arranged to capture images representing a respective field of view (FOV) of at least a portion of an environment in which the cameras are located. In some embodiments, each of the cameras may be arranged within an environment, such as an operating room, where both cameras have at least partial overlapping FOVs. In another embodiment, the cameras may be arranged such that neither of the cameras have overlapping FOVs, in which images captured by both cameras may not include similar (or the same) portions of the environment. For example, the first camera 26 may have a FOV that captures one area of an operating room (e.g., directed towards one wall), which the second camera 27 has another FOV that captures a different non-overlapping area of the operating room (e.g., directed towards an opposite wall of the wall towards which the first camera 26 is directed). In another embodiment, the cameras may have non-overlapping FOVs due to being in different environments, such as one camera being in one room and another camera being in a different, adjacent room. In one embodiment, one or both of the cameras may include a wide-angle lens in order to maximize the observable range of the camera(s). More about FOVs is described herein.

In one embodiment, one of the cameras may be an endoscope that is designed to capture video of a surgical site within a body of a patient during a surgical procedure. In one embodiment, at least one of the cameras camera may be a monocular camera that (e.g., has a single camera sensor that) captures one digital (still) image at a time (e.g., as one video frame). In another embodiment, at least one of the cameras may be a stereoscopic (stereo) camera with two (or more) lenses, each with a separate camera sensor for capturing individual still images (e.g., for producing separate video streams) in order to create 3D video.

The sensor 21 may be any type of electronic device that is configured to detect (or sense) an environment, such as an operating room, and produce sensor data based on the environment. For example, the sensor 21 may include at least one microphone that may be configured to convert acoustical energy caused by sound wave propagation into an input microphone signal. In another embodiment, the sensor may be a proximity sensor, such as an optical sensor that may be configured to detect a presence of one or more objects within the environment and/or a proximity of the sensor to the detected one or more objects. In another embodiment, the sensor may be a temperature sensor that senses an ambient temperature within the environment in which the sensor is located as sensor data.

In some embodiments, the sensor 21 may be a motion sensor, e.g., an inertial measurement unit (IMU), which may be designed to measure a position and/or orientation of the sensor (e.g., with respect to the ambient environment). For example, the IMU may be coupled to or a part of the first camera 26, and may be configured to detect motion of the camera, such as changes to thein the camera's position and/or orientation with respect to a reference point, which may be due to an operator manipulating the camera in order to show a different perspective of the ambient environment. In some embodiments, the motion sensor may be a camera that captures images used by the controller 20 to perform motion tracking operations (e.g., based on changes in the captured images).

The tracking device 22 may be any type of electronic device that is arranged to track and/or detect one or more objects and produce a tracking (or sensor) data associated (e.g., a position with respect to the tracking device) with (e.g., detected) objects. In particular, the tracking device may capture tracking (sensor) data that indicates a position and/or orientation of an object, which may be used by the controller to determine a pose of a tracked object with respect to the tracking device within the environment. For example, the tracking device may be designed to track an object (e.g., its position and/or orientation in a 3D space) with respect to the tracking device, as the object moves within a 3D space, such as an operating room in which both the object and the tracking device may be located. As an example, the tracking device may track an objects spatial transformation with respect to the tracking device, as an object moves from one location to another location. As a result, by tracking an object, the pose of an object that has moved to a new location from an original location may be determined (based on the tracking data), which may therefore be the relative spatial transformation of the object between the first location to the second location. More about using the tracking device to estimate the pose of an object is described herein.

In one embodiment, as the pose of an object may be a transformation that indicates a position (e.g., as a translation vector or matrix) and/or orientation (e.g., as a rotational matrix) of the object with respect to the device 22. In another embodiment, the tracking device may include electronic components, such as one or more processors and memory, which are configurable to track an object within a threshold distance (e.g., radial distance) of the tracking object. In some embodiments, the tracking device may track an object while the object is within the device's FOV and/or line of sight. In another embodiment, the tracking device may be configured to determine the pose of an object that is being tracked by the device 22. In which case, the tracking device may be configured to transmit a pose (as digital data) to the controller 20.

In one embodiment, the tracking device may be a video (e.g., motion tracking) camera. For example, the tracking camera 22 may be a same or similar type of camera as cameras 26 and 27. In another embodiment, the tracking device may be a proximity sensor that, such as an optical sensor (e.g., infrared (IR) sensor), a magnetic sensor, etc. that is arranged to detect the presence, the position, and/or orientation of an object with respect to itself.

In another embodiment, the tracking device may be an electronic device that is capable of detecting a position and/or orientation of a tracking marker. For example, the tracking device may be a radio frequency (RF) position sensor (detector or receiver) that is capable of detecting (measuring) RF signals produced by a RF marker (or transmitter), such as a RF identifier (RFID). Such a device may determine the position of the marker by measuring signal strength of RF signals received from the marker. As another example, the tracking device may be an IR sensor that is capable of tracking an IR marker (or tag) within an environment. In another embodiment, the tracking device may be an electromagnetic sensor that is configured to track electromagnetic markers (or tags).

In another embodiment, the tracking device may be any electronic device that may be configured to track an object that is moving within space based on data received from the object. For example, the tracking device may track the object based on positional data received from the object. In which case, the object may include a positional tracker (e.g., a global positioning system (GPS) device) that produces the positional data, which may be transmitted by the object to the tracking device. As another example, the tracking device may determine an object's position in space based on one or more electronic signals (RF signals) received from the object. For instance, the tracking device may determine the object's position in space based on a signal strength (e.g., received signal strength indicator (RSSI)) in a received RF signal from the object.

The calibration object 23 may be any type of object that may be used for performing extrinsic calibration of one or more cameras of the surgical system 1. In one embodiment, an extrinsic calibration of a camera determines extrinsic parameters, such as a pose of the camera with respect to a reference frame. For example, the first camera 26 may capture an image of the calibration object, which may include a calibration pattern 24, and determines the relative pose of the camera with respect to the calibration pattern and/or vice versa. The camera may be configured to determine the extrinsic parameters based on the image captured by the camera as a transformation from a 3D coordinate system of the camera to a global coordinate system, which may be used to determine a pose of the first camera with respect to a reference frame. In one embodiment, the calibration pattern may be any type of (e.g., visual) pattern, such as a chessboard pattern or a grid pattern.

In one embodiment, the calibration object 23 may be an object that may include a tracking device 25, which may be a same (or similar) type of electronic device as the tracking device 22. In another embodiment, the calibration object may include a tracking marker 92, which may be any type of marker that is designed to be tracked by the tracking device 22. In particular, the tracking marker may be similar (or the same) as at least one object described herein that may be tracked by a tracking device. For example, the marker may be a visual marker, having a unique visual design. As another example, the marker may be any type of tag that is trackable by any type of optical sensor, such as being an IR tag that is detectable by an IR sensor. As another example, the marker may be a tracking tag (e.g., RFID) that is arranged to be tracked by the tracking device 22. As yet another example, the tracking marker may be an electronic device that may be configured to be tracked by another electronic (e.g., tracking) device. For example, the tracking marker may include a GPS device, as described herein.

In some embodiments, the tracking marker 92, the calibration pattern 24, and/or the tracking device 25 may be a part of (or fixedly attached) to the calibration object 23. For example, each (or at least one of) these elements may form one integrated unit with the calibration object 23. In one embodiment, at least some of the elements of the object may be positioned at different locations on (or about) the object 23. For instance, the calibration pattern 24 may be positioned on a side (or front) of the object, while the tracking marker and/or the tracking device 25 may be positioned on (or about) a top side of the object. Specifically, the pattern may be positioned to be visible to a camera that is in front of the object, while the tracking marker 92 may be positioned to be visible by the tracking device 22, which may be located above the object (e.g., on a ceiling of an operating room).

In one embodiment, at least some of the elements of the surgical system may be stationary (e.g., fixedly coupled) within an operating room setting. For example, the first camera 26 may be attached to a wall or an object within the room, while the second camera 27 may be attached to another wall or another object within the room (or a different room). As another example, both cameras may be attached to the same object (e.g., the control tower 3 of the system 1 of FIG. 1), while being directed towards different (or similar) directions. As another example, the tracking device 22 may be attached to a ceiling of the operating room. In another embodiment, the calibration object 23 may be a movable object, and may be separate from (not attached to) at least some of the other elements of the surgical system 1. For example, the calibration object may be separate from the first camera 26, the second camera 27, and/or the tracking device 22. In particular, the object may be sized to be held by a user, such that the user may place the object on a surface (e.g., of a table) at one location, pick up the object, and place the calibration object at a different location on the surface (or on another surface).

The memory (e.g., non-transitory machine-readable storage medium) 90 may be any type of electronic storage device. For example, the memory may include read-only memory, random-access memory, CD-ROMS, DVDs, magnetic tape, optical data storage devices, flash memory devices, and phase change memory. Although illustrated as being separate from the controller 20, the memory may be a part of (e.g., internal memory of) the controller 20. As shown, the memory 90 includes one or more poses 91 of one or more objects, which may be used for camera calibration of one or more cameras of the surgical system 1. In particular, the memory may include poses associated with the calibration object 23. As described herein, the object 23 may include a calibration pattern 24 that may be on (or a part of) a surface (e.g., a front side) of the object, and have a tracking marker 92. In which case, the tracking marker 92 may be on (or a part of) another surface of the object, such as a surface of a top side. The memory may include a pose (or a spatial relative transformation) of the calibration pattern 24 with respect to the tracking marker 92, and/or vice versa. Thus, the poses 91 may include at least one pose that accounts for translation and/or rotation of the calibration pattern with respect to the tracking marker. For example, when the object is square-shaped, a pose may indicate a rotation of 90° about an axis that runs parallel to the front side on which the calibration pattern is attached and the top side of the object on which the marker is attached. In one embodiment, the poses 91 of the elements of the calibration object may be predefined, since the elements may be fixedly attached (e.g., manufactured as one unit).

The controller 20 may be any type of electronic component that is configurable to perform one or more computational operations. For example, the controller may be a special-purpose processor such as an application-specific integrated circuit (ASIC), a general purpose microprocessor, a field-programmable gate array (FPGA), a digital signal controller, or a set of hardware logic structures (e.g., filters, arithmetic logic units, and dedicated state machines). The controller 20 is configured to spatially calibrate one or more cameras of the surgical system using sensor data, such as images captured by the cameras. Such operations allow the surgical system 1 to efficiently and effectively track movement through different fields of view of different cameras. More about how the controller performs calibration operations is described herein.

In one embodiment, at least some of the operations performed by the controller may be performed in response to user input. For example, the controller may be configured to receive user input through one or more input (electronic) devices (not shown), such as a keyboard, mouse, or a peripheral computer device, such as a tablet computer. In another embodiment, user input may be received through a touch-sensitive display (e.g., display 28), which may display a graphical user interface (GUI) with one or more user interface (UI) items, where the device may produce one or more control signals (as the user input) based on a user touching a portion of the display that is presenting a user item. In another embodiment, at least some of the operations may be performed automatically, such as without user intervention. For example, the surgical system 1 may perform calibration operations upon a camera being connected (e.g., for a first time) to the controller 20. As another example, the system may instruct a user (or operator) of the surgical system 1 (e.g., by presenting a notification, such as displaying a pop-up notification on the display 28) to calibrate one or more cameras.

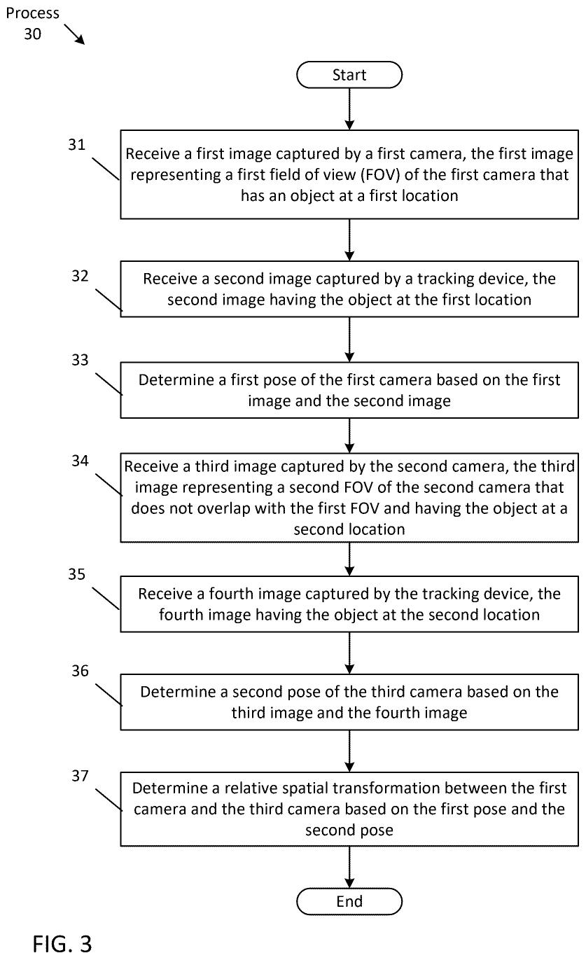

FIGS. 3, 5, and 7 are flowcharts of processes for calibrating one or more cameras of the surgical system 1. In one embodiment, at least some of the operations of at least one of the processes may be performed before the surgical system 1 is used by an operator to perform a surgical (e.g., laparoscopic) procedure upon a patient. In another embodiment, at least some of the operations may be performed intraoperatively (e.g., while an operator is performing a surgical procedure). In another embodiment, at least some of the operations may be performed postoperatively based on image data captured by one or more cameras during a surgical procedure. In some embodiments, at least some of the operations of the processes may be performed by the (e.g., controller 20 of the) surgical system, described herein.

Turning now to FIG. 3, this figure shows a flowchart of a process 30 for an embodiment of calibrating one or more cameras 26 and/or 27 using the tracking device 22. In particular, the process 30 describes calibrating the first camera 26 and the second camera 27 according to the (e.g., calibration pattern 24 of the) calibration object 23 using the tracking device 22, which are located in one or more operating rooms. The controller 20 receives a first image captured by the first camera 26, the first image representing (or including) a first FOV of the first camera 26 that has an object at a first location (at block 31). For example, the calibration object 23 may be placed at the first location (e.g., on a table's surface) in front of the first camera. In one embodiment, the object 23 may be positioned such that the calibration pattern 24 is within the first FOV. For example, the calibration pattern may be at the first location, as described herein. In which case, the image captured by the camera may include the pattern and/or a portion of the environment surrounding the pattern. In one embodiment, once the (e.g., calibration pattern of the) calibration object is placed at the first location, the first camera 26 may capture the first image. In particular, the camera may capture the first image responsive to user input (e.g., a user pressing a button on an input device that transmits a control signal to the controller 20, which causes the camera to capture the image. In another embodiment, the camera may detect that the calibration object 23 has been placed in its FOV (e.g., based on object recognition), and, responsive to detecting the object may capture the first image.

The controller 20 receives a second image captured by the tracking device 22, the second image having the object at the first location (at block 32). In particular, the tracking device may be a tracking camera, which is separate from the first and second cameras, and may have a FOV that includes the first location and therefore includes calibration pattern 24 of the calibration object 23 that is placed at the first location. In one embodiment, both cameras'respective FOVs may include the first location, while both cameras are located at different places within the operating room and are stationary. In which case, both images may include a different perspective of the calibration object. For example, the first camera's FOV may include a forward-facing calibration pattern of the object, whereas the target device's FOV may include an angled top-down (bird's eye) view of the calibration pattern. In another embodiment, the first camera 26 and the tracking device 22 may capture their respective images simultaneously. For example, both cameras may capture their respective images in response to receiving one user input. In another embodiment, the tracking device may capture its image after (or before) the first camera captures its image.

The controller determines a first pose of the first camera based on the first image and the second image (at block 33). In particular, the controller is determining a pose, P_{C1}′, as a relative spatial transformation of the first camera with respect to (e.g., a reference frame of) the tracking device 22. For example, when the tracking device is a camera, an origin of a coordinate system may be defined at a projection center of the camera's lens and one or more axes (orientation) may be defined based on an optical axis and the plane of the camera's imaging sensor. In one embodiment, to determine P_{C1}′, the controller may perform a graph-based approach in which the pose is based on at least two other (e.g., estimated or known) poses of one or more elements within the environment, such as the (e.g., the calibration object 23, the tracking device 22, and the first camera 26 within the operating room). For example, the other poses may be with respect to a same reference frame (or object), within a same coordinate system, where the determined first pose may be determined as a transformation from (or the difference between) one pose to another within a space (e.g., 3D space) of a (e.g., 3D) coordinate system.

In one embodiment, to determine P_{C1}′ the controller may determine the spatial relationship between the first camera and the calibration object, and determine the spatial relationship between the tracking device and the calibration object. For example, the controller may determine a camera (or third) pose, P_{C1}, of the calibration pattern 24 with respect to the first camera, and determine a tracking device (or fourth) pose, P_{TD1}, of the calibration pattern 24 with respect to the tracking device, while the object is at the first location. In some embodiments, one or more poses determined by the controller, such as P_{C1} and/or P_{TD1}, may be inverse spatial transformations of a spatial transformation that is determined by the controller based on each camera's images, as described herein. For instance, the controller may first determine the relative spatial transformation of the calibration pattern 24 with respect to first camera 26 based on one or more images captured by the first camera 26. The controller 20 may define P_{C1} as the inverse of the determined relative spatial transformation between the pattern and the camera in order for the pose to be with respect to the calibration object (e.g., in order to change the reference frame from the camera to the calibration pattern). In which case, both P_{C1} and P_{TD1} may be with respect to the calibration pattern 24 of the object, thereby being within a 3D coordinate system of the pattern.

In one embodiment, either (or both) of poses, P_{C1} and P_{TD1}, may be determined using one or more (predefined) graphical models of the calibration pattern of the object. In one embodiment, the (memory 90 of the) surgical system may include one or more 3D (e.g., computer-aided design (CAD)) models of one or more calibration patterns (and/or calibration objects), each model may be a graphical mathematical coordinate-based representation (e.g., as one or more basis (or B-)splines, such as non-uniform rational basis splines (NURBSs)). In one embodiment, each model may include (or correspond to) one or more different poses (e.g., positions and/or orientations) of a calibration pattern within a 3D coordinate system (e.g., Cartesian coordinate system), with respect to a reference frame, such as at or on a camera. To determine P_{C1}, the controller 20 may be configured to match a 3D model with calibration pattern captured within the first image, which may provide an estimate of a spatial relative transformation of the calibration pattern with respect to the camera. In one embodiment, the pose of the calibration pattern may be estimated based on one or more (e.g., intrinsic) parameters (e.g., determined during a calibration of the camera and/or retrieved from memory 90) and the matching 3D model. In particular, the 3D model may represent the calibration pattern within a 3D model space, and the controller uses the one or more (e.g., intrinsic) parameters of the camera to define the position and orientation of the calibration pattern with respect to a position of the camera. In one embodiment, the controller may apply the intrinsic parameters and the 3D model to (e.g., as input into) a pose model, which produces a pose of the calibration pattern as output. In one embodiment, the controller may determine P_{C1} as an inverse transformation of the estimated pose of the calibration pattern with respect to the first camera 26, as described herein. In another embodiment, the pose model may produce P_{C1}. In another embodiment, the controller 20 may use any known (or future) method to determine P_{C1} from one or more images captured by the first camera 26. For example, the controller may determine P_{C1} based on any extrinsic calibration algorithm, which may be performed by the controller 20. In some embodiments, the controller may determine P_{TD1} by performing one or more of the operations described herein with respect to the determination of P_{C1}.

In one embodiment, with both P_{C1} and P_{TD1}, the controller 20 may be configured to determine the first pose, P_{C1}′, of the first camera with respect to the tracking device. As described herein, the controller 20 may perform a graph-based approach in which the relative spatial transformations are projected within a 3D coordinate system with respect to the same reference, which in this case may be the tracking device 22. For example, each spatial transformation may be a link between a reference (e.g., an origin) and an object (element) that has translated and/or rotated with respect to the reference. In this case, P_{TD1} and P_{C1}′ may be with respect to the same reference, the tracking device 22, while P_{C1} may be a transformation from the first camera to the calibration pattern. In which case, the controller may determine P_{C1}′ based on the relationship between P_{TD1} and P_{C1}. For example, the controller may determine P_{C1}′ as the combination of P_{TD1} and an inverse of P_{C1}. In another embodiment, the controller may be configured to determine P_{C1}′ differently, when P_{C1} and P_{TD1} are with respect to the calibration object. For example, in this case, both poses may be with respect to the calibration pattern 24, and the controller may determine the first pose, P_{C1}′ as a spatial transformation from P_{TD1} to P_{C1}. As a result, the controller may determine P_{C1}′ as the combination of the inverse of P_{TD1} and P_{C1}.

In another embodiment, the controller 20 may determine P_{C1} and/or P_{TD1} by performing an extrinsic calibration method in which intrinsic and/or extrinsic parameters of a camera is estimated using one or more images. For example, the controller may use the calibration pattern of the calibration object captured within the first image by the first camera 26 to determine extrinsic parameters of the camera 26. For example, the controller may estimate PC1 using 3D-2D correspondences between one or more known (e.g., coplanar) 3D points of the calibration pattern 24 (e.g., corners of a chessboard pattern, centroids of circles in a circular grid, etc.), and their corresponding 2D projections onto an image plane of a captured image. As the projections depend on the one or more 3D points, the controller may determine (or estimate) one or more (e.g., intrinsic and/or extrinsic) parameters using an optimization procedure (e.g., a non-linear procedure, such as Levenberg-Marquardt algorithm). From (or using) the extrinsic parameters, the controller may determine P_{C1} as the relative spatial transformation of the calibration object with respect to the camera. In one embodiment, the controller may determine P_{TD1}, of the calibration object with respect to the tracking device using one or more images captured by the tracking device, by performing the extrinsic calibration method.

The controller 20 receives a third image captured by the third camera (e.g., camera 27), the third image representing a second FOV of the second camera that does not overlap with the first FOV and having the calibration object at a second location (at block 34). In particular, the calibration object 23 may be moved (transported) from the first location within the operating room to the second location by. The controller 20 receives a fourth image captured by the tracking camera, the fourth image having the object at the second location (at block 35). Thus, the object may be moved between the two locations, while the first camera 26, the second camera 27, and the target camera 22 remain stationary within the operating room. In which case, the FOV of the target camera may include both the first location and the second location, without having to move (adjusting its location and/or orientation). In one embodiment, the target camera 22 may include a wide-angle lens that expands the FOV of the camara to include both locations. In which case, the target camera 22 and the second camera 27 may capture their respective images simultaneously. In one embodiment, the images of the target camera may be captured sequentially. For example, the first camera 26 and the target camera 22 may capture the first and second images, respectively, before the second camera 27 and the target camera 22 capture the third and fourth images. Conversely, the third and fourth images may be captured before the first and second images. In another embodiment, the calibration object 23 may remain stationary while the first camera 26, the second camera 27, and the target camera 22 capture their respective images. For example, the object may remain stationary at the first location as the first image and second image are captured by the first camera and tracking camera, respectively, and the object may remain stationary at the second location as the third image and the fourth image are captured by the third camera and the tracking camera, respectively.

The controller 20 determines a second pose of the third camera (e.g., the second camera 27) based on the third image and the fourth image (at block 36). For example, the controller 20 may perform similar operations as those described with respect to block 33. For instance, the controller may determine a pose, P_{C2}, of the calibration object 23, which is at the second location, with respect to the second camera 27, and may determine a tracking camera pose, P_{TD2}, of the calibration object 23 at the second location with respect to the tracking camera. The controller may determine the pose of the second camera 27 with respect to the target camera 22, P_{C2}′, based on P_{TD2} and P_{C2}, as described herein. For example, the controller may perform similar operations as described with respect to block 33 to determine P_{C2}′.

The controller determines a relative spatial transformation (e.g., pose) between the first camera and the third camera based on the first pose and the second pose (at block 37). In particular, the controller is determining the relative transformation between the first camera 26 and the second camera 27. In one embodiment, the relative spatial transformation may be one or more extrinsic parameters of at least one of the first and second cameras. For example, the spatial transformation of the first camera, T_{C1}, with respect to the second camera may be the relative transformation indicating a position (e.g., a translation matrix or vector) and/or orientation (e.g., a rotation matrix) of the first camera with respect to the second camera. Similarly, the spatial transformation of the second camera, T_{C2}, may be with respect to the first camera. In one embodiment, either of the relative spatial transformations, such as T_{C1,} may be the result of the combination of P_{C1}′ and P_{C2}′. In some embodiments, since both P_{C1}′ and P_{C2}′ are with respect to the same reference, the tracking camera, at least one of the determined spatial transformations may be inverted. For example, T_{C1} may be the product of the inverse of P_{C1}′ and/or P_{C2}′. As described herein, the determined relative spatial transformations may include similar (or the same) data/information as the determined poses, such as including a translation matrix and/or rotation matrix.

Some embodiments may perform variations to the process 30 described herein. For example, the specific operations of the process may not be performed in the exact order shown and described. The specific operations may not be performed in one continuous series of operations and different specific operations may be performed in different embodiments. As described herein, the controller may receive the first camera 26 and the tracking camera 22 to determine the pose of the first camera with respect to the tracking camera. In another embodiment, each of the cameras may capture one or more images of the calibration object. For example, when determining their respective poses, the controller may apply an extrinsic calibration method. In which case, the method may require multiple images of the object, where in each image the object's position (and/or orientation) may be adjusted. In which case, both cameras may capture one or more images of the object in different positions and/or orientations. In one embodiment, at least some of the operations described in process 30 may be performed sequentially. For example, at least some operations of blocks 31-33 may be performed before the performance of at least some operations of blocks 34-37. In other words, the controller 20 may first determine the pose of the first camera, and then determine the pose of the second camera.

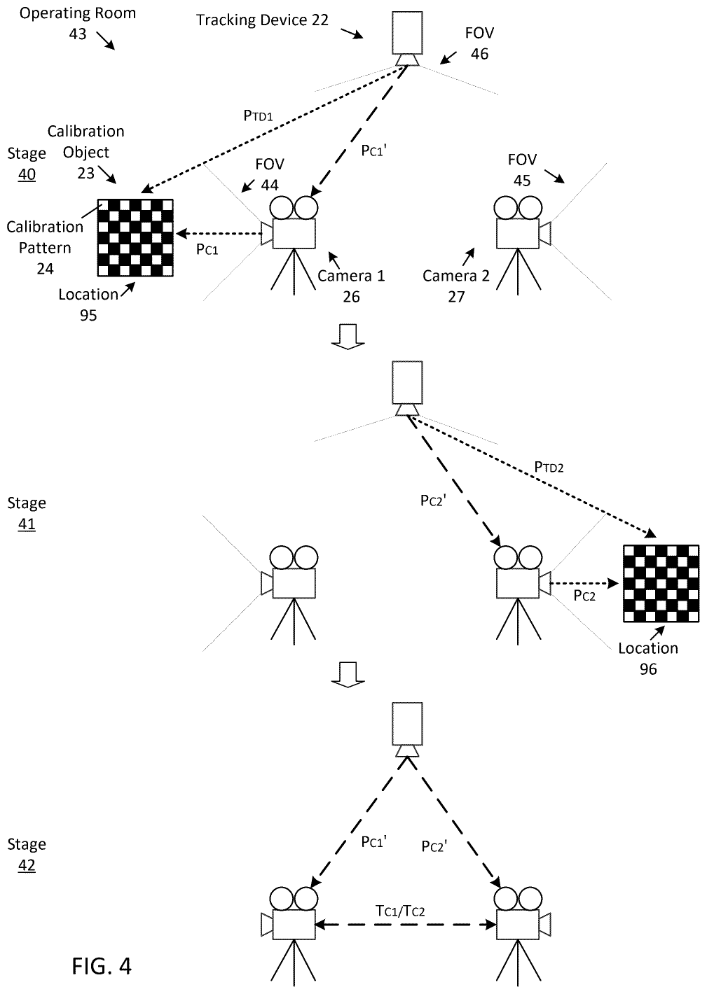

FIG. 4 illustrate several stages 40-42 that show the calibration (e.g., to determine extrinsic parameters) of the first camera 26 and the second camera 27 using the tracking device 22. In particular, this figure shows an illustration of at least some of the operations described in process 30 of FIG. 3. Each stage shows the first and second cameras and the tracking device, which in this example is a tracking camera in an operating room 43. As shown, the first camera 26 and the second camera 27 are each pointing towards opposite directions. Specifically, the first camera has a FOV 44 that is directed in one direction, while the second camera has a FOV 45 directed in an opposite direction. Thus, both FOVs may be non-overlapping, which may be the case when both cameras are placed about the operating room in order to maximize an observable range. In addition, the tracking camera 22 is disposed above the first and second cameras, and having a FOV 46 that includes the first and second cameras. For example, the tracking camera 22 may be fixed to the ceiling of the operating room, while the first and second cameras may be fixed to (e.g., respective) objects of the operating room 43, such as tables, cabinets, etc., or may be stand-alone cameras (e.g., supported by tripods, as shown).

The first stage 40 shows an illustration of estimating the first camera's pose, P_{C1}′, with respect to the tracking device 22, using the calibration object 23. Specifically, this figure is showing that the calibration object 23 is at a (first) location 95, where the object, or more specifically the calibration pattern 24, is in FOV 44 of the first camera 26 and FOV 46 of the tracking device 22.

In addition, this stage is showing that the first camera's pose, P_{C1}′, may be based on P_{C1} and P_{TD1} that may be estimated using images captured by the first camera 26 and the tracking device 22. In particular, this stage is showing a graph-based approach in which two poses are estimated in order to determine P_{C1}′. In particular, P_{C1}, is shown as a link (or projection) that is projecting from the first camera 26 to the (calibration pattern 24 of the) calibration object 23, and P_{TD1} is shown as another link that is projecting from the tracking device 22 to the (calibration pattern 24 of the) calibration object 23. In addition, this stage shows P_{C1}′ as a link between the tracking device 22 and the first camera 26. In one embodiment, P_{C1}′ may be determined graphically based on translational and/or rotational differences between P_{TD1} and P_{C1}. For example, P_{C1}′ may be based on the combination of P_{TD1} and the inverse of P_{C1}, as described herein.

The second stage 41 shows an illustration of estimating the second camera's pose, P_{C2}′, with respect to the tracking camera 22, using the calibration object 23. In particular, this figure is showing that the calibration object has been moved from the location 95 to another (second) location 96, which is now in FOV 45 of the second camera 27. In addition, the new location 96 is still within FOV 46 of the tracking device. Thus, the calibration object 23, which has the calibration pattern 24 has moved from the first location 95 to the second location 96 and is within FOV 45. In one embodiment, the movement may be performed by a person (e.g., a technician). For example, the technician may have picked up the object and moved it between locations. As another example, the object 23 may be mounted on a movable platform (e.g., a cart), and moved between locations, either manually by a person or automatically (e.g., without user intervention), via one or more motors or actuators of the movable cart. In addition, as shown, although the calibration object 23 has moved, the first camera 26, the second camera 27, and the tracking camera 22 have remained in place (stationary) within the operating room 43.

In addition, the second stage is showing the poses as graphical links between the second camera 27 and the calibration object 23, and the tracking device 22 and the calibration object. In particular, this figure is showing P_{C2}, between the calibration (pattern of the calibration) object and the second camera 27, and P_{TD2}, between the calibration object and the tracking device 22. In addition, this stage shows P_{C2}′, as a link between the second camera 27 and the tracking device 22, which may be graphically determined based on the relationship between P_{C2}′ and P_{TD2}, as described herein. In one embodiment, at least some of the poses shown in this stage may be determined in a similar manner as the poses within the first stage 40 described herein.

The third stage 42 is showing the result of determining poses P_{C1}′ and P_{C2}′. In particular, it is showing the spatial relative transformations of both the first camera 26 and the second camera 27, as links from the tracking camera, both being in the same coordinate system (e.g., with respect to the tracking camera 22). As a result, the controller 20 may determine spatial relative transformations T_{C1} and T_{C2} between both cameras based on differences between the two poses. For instance, T_{C1} may be the spatial relative transformation of the first camera with respect to the second camera, while T_{C2} may be the spatial relative transformation of the second camera with respect to the first camera

As shown herein, FOV 46 of the tracking camera 22 includes both locations 95 and 96, and the first cameras 26 and the second camera 27. In one embodiment, the FOV 46 may not include one or both of the cameras, but may only have the locations of the object in its field of view. In another embodiment, the positions and/or orientations of the cameras may be different, such as the cameras may be positioned in a circular fashion (e.g., when there are two or more cameras of which the surgical system is calibrating).

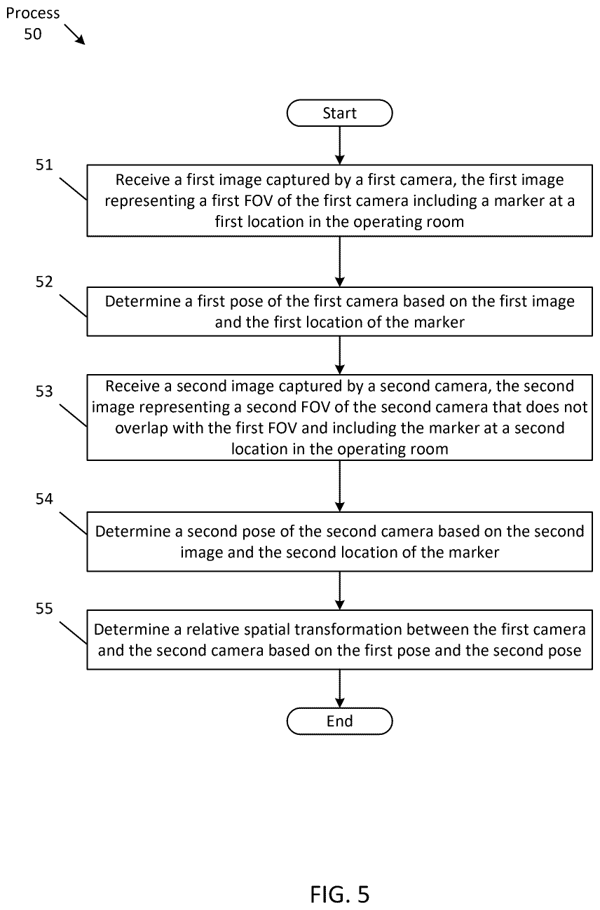

FIG. 5 is a flowchart of a process 50 for an embodiment of calibrating one or more cameras, such as cameras 26 and/or 27, using a tracking device 22 and a tracking marker 92 that may be a part of the calibration object 23. Specifically, the tracking marker 92 may be a part of (fixedly coupled) to the calibration object 23 that includes the calibration pattern 24. The process 50 begins by the controller 20 receiving a first image captured by the first camera 26, the first image representing a first FOV of the first camera including the marker 92 at a first location in the operating room (at block 51). In particular, the calibration object may be at the first location (e.g., placed at the location by a user), where the captured image include at least a portion of the calibration pattern. In one embodiment, the tracking marker 92 may be disposed on a different side (or surface) than the calibration pattern, and therefore may not be within the FOV of the first camera. As a result, the captured image may not include the tracking marker.

The controller 20 determines the first pose, P_{C1}′, of the first camera 26 based on the first image and the first location of the marker (at block 52). In particular, the controller may determine P_{C1}′ based on one or more poses that are estimated using (at least) the first image, and/or a detection of the marker. For example, the controller may determine the pose of the calibration pattern 24 with respect to the first camera, P_{C1}, while the object 23 that is at the first location, as described herein. In addition, the controller may determine the pose P_{TD1} of the calibration pattern of the calibration object with respect to the tracking device 22. In one embodiment, to do this, the controller may determine a spatial relationship between the tracking device 22 and the tracking marker 92. In one embodiment, the tracking device may be a proximity (or location detection) sensor that is configured to detect a positional data (e.g., a position and/or orientation) of the tracking marker. In particular, the controller may be configured to detect (determine) the location (and/or orientation) of the tracking marker 92 with respect to the tracking device using tracking/sensor data produced by the device. For example, when the tracking marker includes a RF transmitter (or RF tag, such as a RFID) and the tracking device includes a RF sensor, the RF sensor may be arranged to sense RF signals produced (or reflected off of) the RF tag and produce sensor data from the signals. In another embodiment, the tracking device may be an IR sensor, and the marker may be an IR tag. In which case, the tracking device may produce sensor data based on infrared signals produced by the IR sensor that are reflected back from the IR tag that indicates positional characteristics of the marker. In one embodiment, the sensor data may indicate the position and/or orientation of the marker with respect to the tracking device. The controller may be configured to determine (estimate) a pose, P_{TD1}′, of the tracking marker with respect to the tracking device 22, according to the determined location of the marker (e.g., using the sensor data). In which case, the controller may use this information to estimate P_{TD1}′ of the tracking marker 92 with respect to the tracking device (e.g., by producing a transformation matrix that indicates a translation and/or rotation of the marker with respect to the tracking device). In one embodiment, P_{TD1}′ may be an inverse transformation of the pose of the tracking marker 92 that is determined with respect to the tracking device, as described herein.

In another embodiment, P_{TD1}′ may be estimated using one or more images, as described herein. For example, when the tracking device 22 is a tracking camera, it may be arranged to capture one or more images of the tracking marker 92, which may include one or more visible patterns (or objects), and the controller may determine P_{TD1}', as described herein. This may be the case when the calibration pattern 24 is not within the field of view of the tracking device 22.

As described herein, the tracking marker 92 may be at a different location on the calibration object, than the calibration pattern 24 that may be used to determine P_{C1}. For example, the marker may be attached to a top surface of the object, whereas the calibration pattern may be attached to a front surface of the object. In which case, the controller may be configured to determine P_{TD1} by accounting for the relative spatial relationship between the marker and the pattern. For example, the controller may retrieve (or receive) a pose, P_{TM}, of the tracking marker with respect to the calibration pattern (e.g., from the poses 91 in memory 90), and may adjust P_{TD1}′ according to P_{TM}. As described herein, this adjustment may be performed using a graph-based approach. As a result, the controller may estimate P_{C1}′ as the combination of P_{TD1} and P_{C1}, as described herein.

The controller 20 receives a second image captured by the second camera 27, the second image representing a second FOV of the second camera that does not overlap with the first FOV of the first camera, and including the marker at a second location in the operating room (at block 53). In particular, the object to which the marker is fixedly attached may be moved from one location to another in the operating room, as described herein.

The controller 20 determines a second pose, P_{C2}′, of the second camera 27 based on the second image and the second location of the marker (at block 54). In one embodiment, the controller may perform similar operations as described for block 52 to determine P_{C2}′. For example, the controller may determine a pose, P_{C2}, of the calibration pattern 24 of the object that is at the second location with respect to the second camera, and determine P_{TD2} of the calibration pattern 24 with respect to the tracking device 22. In one embodiment, P_{TD2} may be estimated based on tracking data produced by the tracking device, as the (e.g., calibration object of the) tracking marker is moved from the first location to the second location. More about tracking markers is described herein. As a result, the controller may be configured to estimate a pose, P_{TD2}′, of the tracking marker with respect to the device 22, and may adjust this pose with respect to P_{TM}.

The controller 20 determines the relative spatial transformation between the first camera 26 and the second camera 27 based on the first pose, P_{C1}′ and the second pose, P_{C2}′ (at block 55). Thus, in this embodiment, the controller determines the relative spatial transformation using a location of a tracking marker on the calibration object, while the cameras and the tracking device remain stationary as the (e.g., calibration object of the) marker is moved between locations within the operating room.

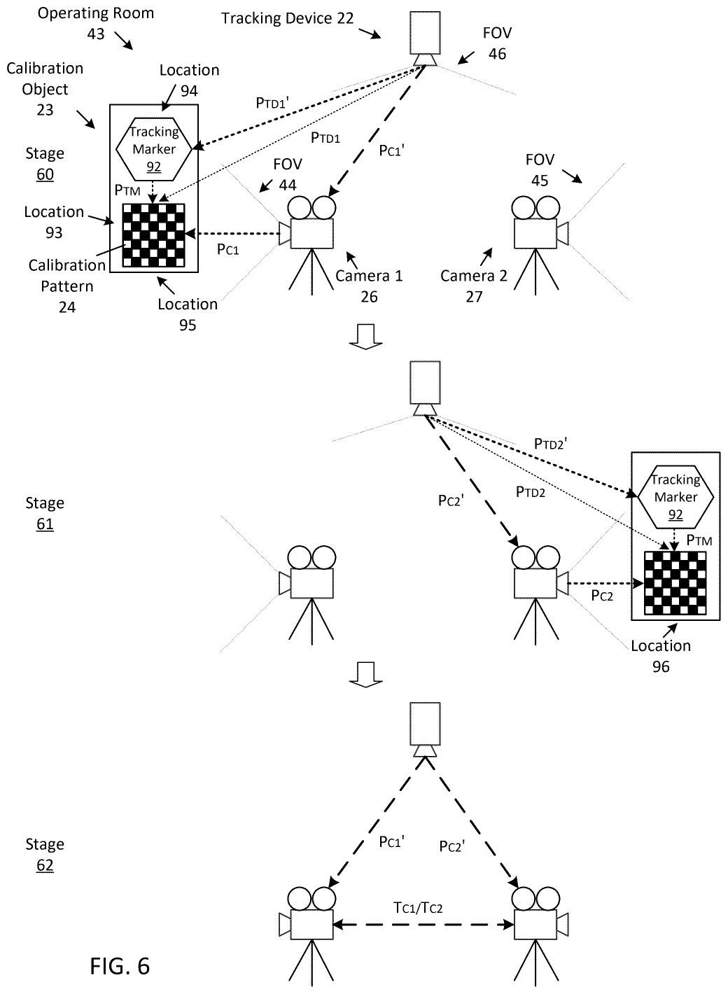

FIG. 6 illustrate several stages 60-62 that show the calibration of the first camera 26 and the second camera 27 using the tracking device 22 and the tracking marker 92 of the calibration object 23. In particular, this figure shows an illustration of at least some of the operations described in process 50 of FIG. 5. Each of these stages shows the first camera 26, the second camera 27, and the tracking device 22, which may be any type of electronic device that is capable of detecting a location of an object (and/or a marker) within space, such as an IR sensor or a camera. In addition, this figure shows the calibration object 23 includes the calibration pattern 24 and the tracking marker 92. In particular, this is showing that the calibration pattern 24 is at a location 93 on (or about) the calibration object 23, while the tracking marker 92 is at another location 94. For example, the calibration pattern may be on a front surface of the object, whereas the marker may be on a top surface of the object, as described herein.

The first stage 60 shows an illustration of estimating P_{C1}′, which may be based on (e.g., spatial differences) between P_{C1} and P_{TD1}, as described herein. In particular, this figure is showing that the calibration object 23 is at the location 95, which includes the calibration pattern 24 that is in the FOV 44 of the first camera 26. In addition, the tracking marker 92, such as an IR tag, may not be within FOV 44, or may at least partially be within FOV 44. This stage also shows that the tracking marker 92 is within the FOV 46 of the tracking device 22. This may be the case when the tracking device is a tracking camera. In another embodiment, FOV 46 may represent a (e.g., radial) distance and/or line of sight, within which the tracking device may track one or more objects, such as the marker 92. For example, when the tracking device is a proximity sensor, FOV 46 may be a distance within which the sensor may detect the proximity of objects.

This first stage 60 illustrates the poses that are determined by the surgical system in order to estimate P_{C1}′ (e.g., using a graph-based approach, as described herein). In particular, this figure is showing P_{C1} between the calibration pattern 24 and the first camera 26, and shows P_{TD1} between the calibration pattern 24 and the tracking device 22, which is determined based on the detected location of the tracking marker 92 by the tracking device 22, as described herein. Since, however, the calibration pattern may not be in the line of sight (or within the FOV 46) of the tracking device 22, for example, the controller determines P_{TD1} based on the detected location of the tracking marker 92. To do this, the controller determines P_{TD1}′, which is the pose of the tracking marker 92 with respect to the tracking device 22, since the tracking marker may be within the FOV 46 of the device 22. This figure also shows P_{TM} between the calibration pattern 24 and the tracking marker 92. In this case, the controller may determine P_{TD1} graphically as a combination of (e.g., the product of) P_{TD1}′ and P_{TM}.

The second stage 61 shows an illustration of estimating P_{C2}′,, which may be based on the spatial relationship between P_{C2} and P_{TD2}, as described herein. In particular, this figure is showing that the calibration object 23 has been moved from the first location 95 to location 96, and is in the FOV 45 of the second camera 27. In addition, the tracking marker 92 may be within FOV 46 of the tracking device. In one embodiment, the controller may perform similar operations as described for the first stage 60. In one embodiment, the tracking marker 92 may remain within FOV 46 as it moves from the first location 95 to the second location 96.

This stage is showing P_{C2} between the calibration pattern 24 and the second camera 27, and shows P_{TD2} between the calibration pattern 24 and the tracking device 22. In one embodiment, in order to determine P_{TD2}, the controller may determine the relationship between the tracking marker 92 and the tracking device 22. Thus, this figure shows P_{TD2}′ between the tracking marker 92 and the tracking device 22, which may be determined based on a detected position of the marker by the tracking device, as described herein. Since the tracking marker 92 and the calibration pattern are fixedly coupled to the calibration object 23, the relative spatial transformation, P_{TM} remains the same as shown in the first stage 60. In one embodiment, P_{TD2}′ may be different than P_{TD1}′, since the (e.g., calibration object of the) tracking marker has moved between location 95 and location 96. The controller than determines P_{TD2} as a combination of P_{TD2}′ and P_{TM}, as described herein. In some embodiments, the tracking marker 92 may remain within the FOV (e.g., line of sight) 46 of the tracking device 22 at (and/or between) locations 95 and 96. In another embodiment, the FOV 46 may be a threshold distance, such as a radial distance, from the tracking device 22 in which both locations may be located.

The third stage 62 is showing the result of determining poses P_{C1}′ and P_{C2}′, in which the controller may determine the spatial relative transformation of the first camera with respect to the second camera, T_{C1}, or the spatial relative transformation of the second camera with respect to the first camera, T_{C2}, using a graph-based approach by identifying the translations and/or rotations between P_{C1}′ and P_{C2}′, as described herein.

The calibration operations described in FIGS. 5 and 6 is a calibration method determine the spatial relationship between at least two cameras of the surgical system 1 using the tracking device to track the location of a marker. In one embodiment, this method allows for high accuracy and tolerance with respect to the ambient conditions (e.g., illumination within) an operating room by the tracking device being able to effectively track the marker from the first location to the second location.

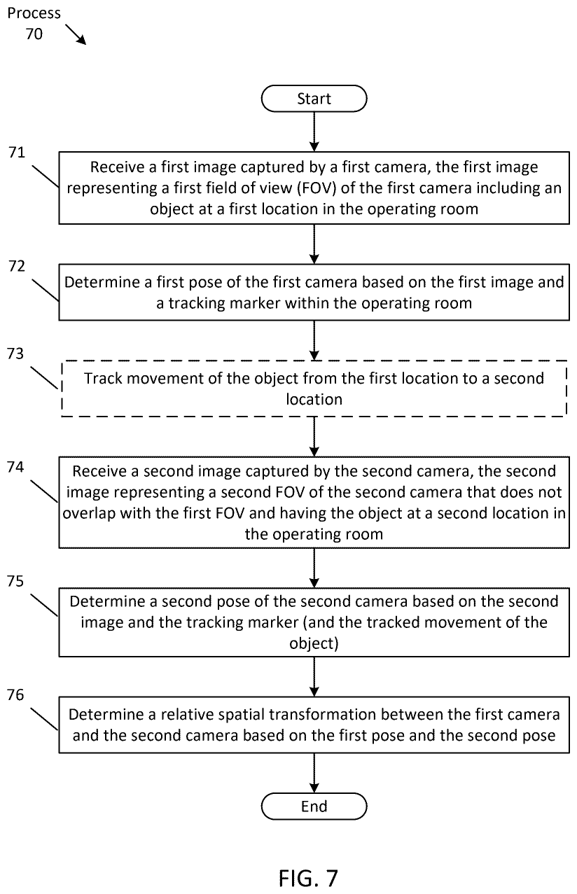

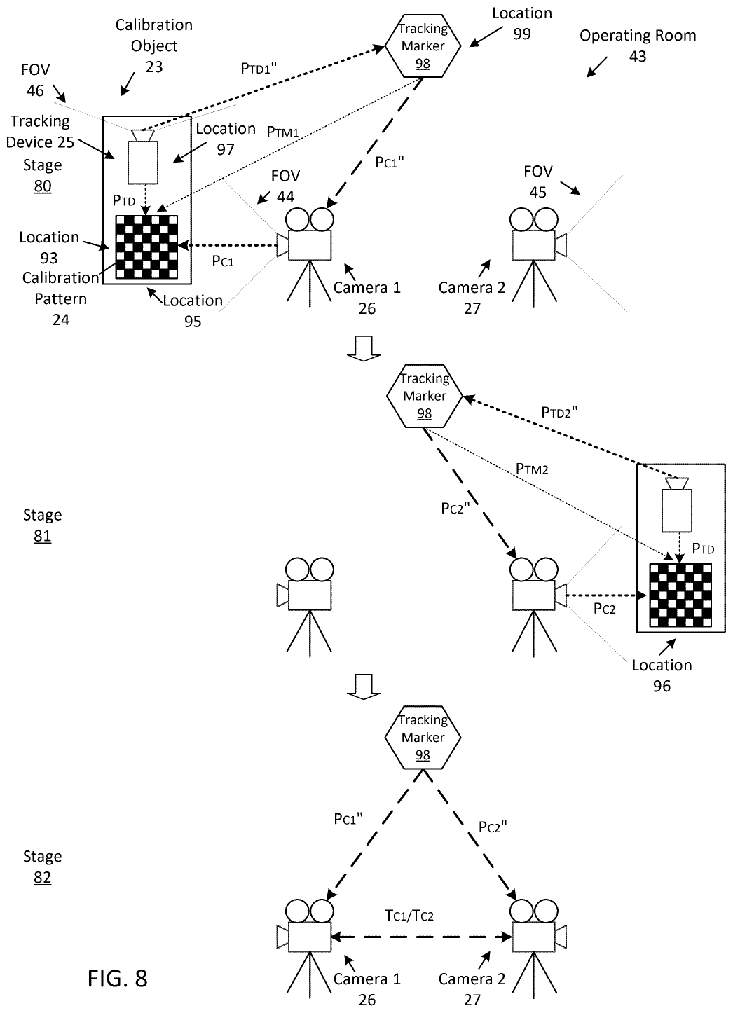

The calibration methods described thus far include the estimation of relative spatial transformations between spatially distributed static image sensors (e.g., RGB video cameras or RGBD depth sensors), In particular, both of the calibration methods described in FIGS. 3-6 may be “outside-in” tracking calibration methods in which the sensors, such as the tracking device 22, which is static (e.g., stationary) within the operating room, tracks the (e.g., marker 92 of the) calibration object 23 between two or more locations. In another embodiment, the surgical system may be configured to perform one or more “inside-out” tracking calibration methods in which one or more tracking devices are movable within the operating room in order to track movement of the tracking device based on detected positional changes of one or more (e.g., stationary) objects with respect to the tracking device. In particular, FIGS. 7 and 8 relate to such an inside-out tracking calibration method.

Turning now to FIG. 7, this figure shows a flowchart of a process 70 for an embodiment of performing an inside-out calibration of one or more cameras, such as the first camera 26 and/or the second camera 27, using a tracking device and a tracking marker. Specifically, the process 70 describes calibrating cameras using the tracking device 25 that is coupled to (or a part of) the calibration object 23 in order to track the movement of the (e.g., calibration pattern 24 of the) calibration object 23 by detecting relative spatial changes of the tracking device 25 with respect to a separate tracking marker (e.g., tracking marker 98, as shown in FIG. 8), which may be separate from the calibration object 23. In one embodiment, the tracking marker 98 may be a static (stationary) object within the operating room, whereas the calibration object 23, which includes the tracking device 25, may be moveable.

The process 70 begins by the controller receiving a first image captured by the first camera 26, the first image representing a first FOV of the first camera 26 including an object at a first location in the operating room (at block 71). Thus, as described herein, the FOV of the first camera may include the calibration object 23 at the first location, which may be placed there by a user. The controller determines a first pose, P_{C1}″, of the first camera 26 based on the first image and the tracking marker 98 within the operating room (at block 72). In particular, the controller 20 may determine P_{C1}″, which may be a pose of the first camera 26 with respect to the tracking marker 98 based on one or more estimated poses. Thus, unlike at least some of the other calibration methods described herein in which the controller determines the first pose (e.g., P_{C1}′) of the first camera 26 with respect to the tracking device 22, the controller 20 may determine the pose of the first (and second) camera with respect to the tracking marker 98. As a result, P_{C1}″ may be determined based on a detection of a tracking marker by a tracking device 25 of the calibration object that may be at the first location. More about these operations is describe herein.

In one embodiment, the controller 20 may determine P_{C1}″ as follows. Specifically, the controller 20 may determine P_{C1} using (at least) the first image, as described herein. The controller may determine P_{C1}″ based on (or as a combination of) P_{C1} and a pose, P_{TM1}, of the (e.g., calibration pattern 24 of the) calibration object 23 with respect to the tracking marker 98. In particular, P_{TM1} may represent the spatial relative transformation of the calibration object 23 while at the first location from the tracking marker 98 at its particular (stationary) location within the operating room. In one embodiment, the transformation between the calibration object and the tracking marker 98 may change as the object's location within the operating room changes. More about the transformation changing is describe herein.

In one embodiment, to determine P_{TM1}, the controller may determine a pose, P_{TD1}″, of the tracking marker 98 with respect to the tracking device 25. Specifically, the controller detects, using the tracking device 25, the tracking marker 98 while the calibration object is at the first location, and determines P_{TD1}″ based on the detection of the tracking marker while the (e.g., tracking device 25 of the) calibration object is at the first location. In one embodiment, the tracking device 25 may produce sensor data upon detecting the tracking marker indicating its position and/or orientation within the operating room, and using this data, the controller 20 may determine P_{TD1}″. In some embodiments, the controller may perform one or more operations described herein to determine P_{TD1}″ from sensor data. For example, when the tracking device 25 is a tracking camera, the tracking marker 98 is detected based on one or more images captured by the tracking camera, and from those one or more images the controller may determine the pose of the tracking marker with respect to the tracking device 25, as described herein.

In one embodiment, to produce P_{TM1}, the controller may be configured to account for the relative spatial relationship between the tracking device 25 and the calibration pattern 24 of the calibration object 23. In one embodiment, the calibration pattern 24 is at one location on the calibration object 23, while the tracking device 22 is at another location on the calibration object 23. For instance, the tracking device may be at or near a location on the top surface of the calibration object in order to have an upward FOV (or line of sight) in order to observe the tracking marker that may be above the calibration object, whereas the location of the pattern may be towards a side of the object. As a result, the controller may retrieve a pose, P_{TD}, of the calibration pattern (e.g., from the poses 91 in memory 90) with respect to the tracking device 25. In one embodiment, the controller may estimate P_{TM1} by adjusting P_{TD1}″ to account for the transformation of P_{TD}. For example, P_{TM1} may be based on a combination of (an inverse of) P_{TD1}″ and P_{TD}. In one embodiment, with P_{TM1} and P_{C1}, the controller may be configured to determine P_{C1}″ as a combination of P_{TM1} and (an inverse of) P_{C1}.

Returning to process 70, the controller 20 may track movement of the object from the first location to a second (different) location (at block 73). In particular, the controller may use the tracking device 25 that is coupled to the calibration object 23 to track movement based on sensor data produced by the device. For example, as a user picks up the calibration object and moves it to the different location, the tracking device may be (e.g., periodically, such as every second) capturing sensor data that indicates a change in location and/or orientation of the tracking device (and/or object). As an example, when the tracking device 25 is a motion camera that is arranged to capture one or more images of the environment, the controller may be configured to perform a camera motion tracking algorithm (e.g., a Simultaneous Localization and Mapping (SLAM) algorithm, a Visual Odometry (VO) algorithm, etc.) for tracking the movement of the device based on movement of one or more points within a succession of one or more video frames (images) captured by the camera. As described herein, a relative spatial transformation of the tracking device at the second location may be based on the tracked movement of the object from the first location in space with respect to the tracking marker.

The controller 20 receives a second image captured by the second camera 27, the second image representing a second FOV of the second camera that does not overlap with the first FOV and having the object at a second location in the operating room (at block 74). For example, the calibration object 23 may be manually moved by a user from the first location within the operating room to another location. In another embodiment, the object may be moved autonomously (e.g., by an autonomous robot within the room). In one embodiment, the tracking marker 98 may remain within the tracking device's field of view while the object is moved.

The controller 20 determines a second pose, P_{C2}″, of the second camera 27 based on the second image and the tracking marker (at block 75). In one embodiment, the controller may perform at least some of the operations described herein, such as with respect to block 72 of this process, to determine P_{C2}″. For example, the controller may determine the pose of the calibration pattern 24 of the object at the second location, P_{C2}, with respect to the second camera, and determine P_{TM2}, which is the pose of the calibration pattern 24 of the calibration object, while the object is at the second location, with respect to the tracking marker 98. In one embodiment, the controller may determine P_{TM2} by performing at least some of the operations described herein with respect to P_{TM1}. In one embodiment, the controller may determine P_{TM2} based on the detected movement of the tracking device. For example, the controller may use the tracking data, which may indicate changes in location and/or orientation of the tracking device as it moves in space, and that was produced by the tracking device while the object was moved from the first location to the second location to determine P_{TD2}″. In which case, P_{TD2}″ may be the relative spatial transformation of the tracking device 25 from the first location to the second location. In another embodiment, the P_{TD2}″ may be estimated based on the tracking data and based on sensor data produced by the tracking device 25 at the second location. For instance, the tracking data may indicate one or more translational parameters from the first location to the second location with respect to the tracking marker and/or one or more rotational parameters, while other translational and/or rotational parameters may be determined based on sensor data captured by the tracking device at the second location. The controller may determine P_{C2}″ based on P_{C2} and P_{TM2}, as described herein. The controller determines a relative spatial transformation between the first camera 26 and the second camera 27 based on the first pose, P_{C1}″, and the second pose, P_{C2}″ (at block 76). For example, to determine T_{C1} using the graph-based approach, the controller may determine the positional differences (or changes) from P_{C2}″ to P_{C1}″, as described herein. In one embodiment, the controller may be configured to determine one or more extrinsic parameters of the first and second cameras based on the tracked movement of the cameras. For example, the tracked movement may indicate a distance between the first camera 26 and the second camera 27. In which case, the controller may determine one or more translational parameters of a translation vector associated with T_{C1} and T_{C2} based on the determined distance.

FIG. 8 illustrate several stages 80-82 that show calibrating the first camera 26 and the second camera 27 using the tracking device 25 of the calibration object 23 to detect the tracking marker 98. In particular this figure shows an illustration of at least some of the operations described in process 70 of FIG. 7. Each of these stages shows the first camera 26, the second camera 27, and the tracking marker 98 that are disposed within one or more operating rooms 43. Specifically, the tracking marker 98 is shown at location 99, which is above both of the cameras. For instance, the tracking marker 98 may be attached to a ceiling of the operating room, while the two cameras are supported on the floor of the room. In another embodiment, the tracking marker 98 may be located at a different location within the room (e.g., on a wall of the room). In addition, this figure shows the calibration object 23 that includes the calibration pattern 24 at a first location 93 on the object 23 and the tracking device 25 that is at another location 97 on (or at) the object. In one embodiment, each of these locations may be on different surfaces of the object (e.g., the location 93 being on a front-facing surface, whereas the location 97 is on a top-facing surface).

The first stage 80 shows an illustration of estimating the first camera's pose, P_{C1}″, with respect to the tracking marker 98, using the tracking device 25 of the calibration object 23. Specifically, this figure is showing the calibration object at the location 95, where the calibration pattern 24 is in FOV 44 of the first camera 26. In addition, the FOV 46 of the tracking device 25 may include the tracking marker 98, such that the tracking device may be able to detect the marker. This stage also shows poses, e.g., as links between elements within the operating room, which are determined by the controller 20 to estimate P_{C1}″ that is shown as a link between the tracking marker 98 and the first camera 26. In particular, this stage is showing P_{C1} between the calibration pattern 24 and the first camera 26, which is determined based on images captured by the first camera 26, as described herein. In addition, this example is also showing P_{TD1}″ that is the pose of the tracking marker 98 with respect to the tracking device, which may be determined by the controller based on sensor data from the tracking device 25, as described herein. P_{TD} is also shown and may be the pose of the calibration pattern 24 with respect to the tracking device 25. In one embodiment, the controller 20 may use these two poses to determine P_{TM1}, which is shown as a link between the tracking marker 98 and the calibration pattern. As described herein, the controller may graph these poses in a coordinate system of the calibration pattern 24 to determine P_{C1}″, which may be based on the combination of P_{TM1} and P_{C1}.

The second stage 81 shows an illustration of estimating P_{C2}″, which is the estimated pose of the second camera 27 with respect to the tracking marker 98, which may be performed while the calibration object 23 is at a new location Specifically, this figure is showing that the object 23 has been moved from the location 95 to location 96, which is within the FOV 45 of the second camera 27. As described herein, the controller 20 may perform similar operations as those described with respect to the first 80 to determine one or more poses to estimate P_{C2}″. For example, this figure is showing the pose of the second camera 27, P_{C2}, with respect to the calibration pattern 24. This figure also shows P_{TD2}″ between the tracking device 25 and the tracking marker 98, which may be estimated based on sensor data captured by the tracking device 25 at the location 96 and/or based on tracking data captured by the tracking device as it is moved from the first location 95 to the second location 96. In one embodiment, the controller may adjust P_{TD2}″ according to P_{TD} in order to estimate P_{TM2}. The controller than determines P_{C2}″ based on P_{C2} and P_{TM2}, as described herein.

The third stage 82 is showing the result of determining poses P_{C1}″ and P_{C2}″, in which the controller may determine the spatial relative transformation of the first camera with respect to the second camera, T_{C1}, or the spatial relative transformation of the second camera with respect to the first camera, T_{C2}, using a graph-based approach by identifying the translations and/or rotations between P_{C1}″ and P_{C2}″, as described herein.

Some embodiments may perform variations to the process 30 described herein. For example, the specific operations of the process may not be performed in the exact order shown and described. The specific operations may not be performed in one continuous series of operations and different specific operations may be performed in different embodiments. In one embodiment, at least some of the operations described herein may be performed once in order to calibrate two or more cameras. For example, at least some operations and/or at least some elements with dashed boundaries illustrated herein may be optional, as described herein. In another embodiment, the process 70 may be performed in order to calibrate the first camera 26 and the second camera 27 at an initial setup of the cameras. Once calibrated, the surgical system 1 may use the estimated extrinsic parameters (relative spatial transformation) while performing visual processing operations, such as motion detection. In another embodiment, at least some of the operations may be performed periodically in order to re-calibrate one or more cameras over a period of time. For instance, over time, one or more parameters of a camera may drift (e.g., gradually change). As a result, the surgical system may perform at least some of these operations after a period of time in order to ensure that the parameters are accurate.

As described herein, the surgical system may perform at least one of the processes 30, 50, or 70 to calibrate one or more cameras. In one embodiment, the surgical system may perform two or more calibration methods described herein in order to optimize estimated extrinsic parameters. For instance, the surgical system may perform at least some of the operations of processes 30 and 70 of FIGS. 3 and 7, respectively, and may determine the relative spatial transformations between the first camera 26 and the second camera 27 based on two sets of transformations for each camera. In one embodiment, when performing two or more calibration methods, the surgical system may average the estimated relative spatial transformations between cameras.

In one embodiment, at least some of the operations may be performed in order to calibrate the first camera 26 and the second camera 27 of the surgical system 1. In particular, the surgical system may perform one or more calibration operations describe herein to calibrate three or more cameras. In which case, the surgical system 1 may be configured to estimate the relative spatial transformation between pairs of cameras. For example, in the case of three cameras, cameras “A”, “B”, and “C”, the surgical system may determine a first pair of relative spatial transformations between cameras A and B, a second pair of relative spatial transformations between cameras A and C, and/or a third pair of relative spatial transformations between cameras B and C, using one or more of the calibration methods described herein. In some embodiments, the surgical system may determine each pair of spatial transformations between the cameras within a same coordinate system, using the graph-based approach, as describe herein.

In one embodiment, the operations described herein may allow the surgical system to calibrate cameras with non-overlapping fields of view. In another embodiment, at least some of the operations describe herein may be used to calibrate cameras with at least partial overlapping fields of view.

As described herein, the tracking devices 22 and 25 are arranged to detect the tracking markers 92 and 98, respectively, in order to track movement of the calibration object. In particular, the tracking devices may track movement by capturing sensor data at locations at which the calibration object is placed. In another embodiment, the tracking devices may track the movement of the calibration object as it is moved about the operating room and/or between one or more operating rooms. As an example, referring to FIG. 8, the tracking device 25 may track movement (e.g., of the calibration object), as the object is moved between locations (e.g., carried by a user). This may allow cameras that are within different operating rooms to be calibrated with respect to each other. For instance, the surgical system may use the tracking device 25 to track movement from the first location 95 that is in one room to the second location 96 that is in another room. In which case, the surgical system may account for the distance between the two cameras using the tracked movement of the object. In one embodiment, the surgical system may use sensor data from either (or both) of the tracking devices 22 and 25 to track movement of the calibration object to more accurately and effectively estimate one or more poses described herein.

In one embodiment, at least some of the operations described herein, may be performed in “real-time”, meaning that the operations may be performed by the surgical system as one or more images are captured by one or more cameras that are to be calibrated. In one embodiment, the surgical system may provide the user with feedback as the user is calibrating the cameras. As an example, turning to FIG. 8, once the user places the calibration object at location 95, the surgical system may provide a notification to the user indicating whether or not the object is within the FOV of the first camera. For example, once placed, the surgical system may provide a pop-up notification indicating whether or not the calibration object needs to be adjusted. Once P_{C1}″ is estimated, the surgical system may provide a notification alerting the user to move the object to the second location 96. In this case, the surgical system may output an audible notification using the speaker 29, saying “Please move the calibration pattern in front of the next camera.” Providing feedback may ensure that the surgical system is efficiently and effectively calibrated.

As described herein, the surgical system 1 may be configured to estimate a pose of an object with respect to another object based on sensor data. In another embodiment, an estimated (or overall) pose may be based on one or more estimated poses, such that the estimated overall pose may be an average of the one or more poses. For example, referring to FIG. 4, the controller may be configured to estimate one or more P_{C1}'s. In this case, the surgical system 1 may estimate multiple P_{C1}'s based on changes to other poses that are used to estimate a P_{C1}′. For example, the surgical system may estimate a first P_{C1}′ while the calibration pattern is in a first orientation with respect to the camera 1, and may estimate a second P_{C1}′ while the calibration pattern is in a second orientation with respect to the camera 1. In one embodiment, the calibration pattern may be at the same location 95, while having different orientations. The resulting P_{C1}′ may be based on (e.g., an average) of the first P_{C1}′ and the second P_{C1}′. In one embodiment, estimating poses based on an average one or more poses may reduce noisy data and may produce a better overall pose estimate.

As previously explained, an embodiment of the disclosure may be a non-transitory machine-readable medium (such as microelectronic memory) having stored thereon instructions, which program one or more data processing components (generically referred to here as a “processor”) to automatically (e.g., without user intervention) calibrate one or more cameras using one or more images, as described herein. In other embodiments, some of these operations might be performed by specific hardware components that contain hardwired logic. Those operations might alternatively be performed by any combination of programmed data processing components and fixed hardwired circuit components.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

While certain embodiments have been described and shown in the accompanying drawings, it is to be understood that such embodiments are merely illustrative of and not restrictive on the broad disclosure, and that the disclosure is not limited to the specific constructions and arrangements shown and described, since various other modifications may occur to those of ordinary skill in the art. The description is thus to be regarded as illustrative instead of limiting.

In some embodiments, this disclosure may include the language, for example, “at least one of [element A] and [element B].” This language may refer to one or more of the elements. For example, “at least one of A and B” may refer to “A,” “B,” or “A and B.” Specifically, “at least one of A and B” may refer to “at least one of A and at least one of B,” or “at least of either A or B.” In some embodiments, this disclosure may include the language, for example, “[element A], [element B], and/or [element C].” This language may refer to either of the elements or any combination thereof. For instance, “A, B, and/or C” may refer to “A,” “B,” “C,” “A and B,” “A and C,” “B and C,” or “A, B, and C.”

What is claimed is:

1. A method performed by a surgical system that includes a first camera and a second camera that are located within an operating room, the method comprising:
receiving a first image captured by the first camera, the first image representing a first field of view (FOV) of the first camera including a marker at a first location in the operating room;
determining a first pose of the first camera based on the first image and the first location of the marker;
receiving a second image captured by the second camera, the second image representing a second FOV of the second camera that does not overlap with the first FOV and including the marker at a second location in the operating room;
determining a second pose of the second camera based on the second image and the second location of the marker; and
determining a relative spatial transformation between the first camera and the second camera based on the first pose and the second pose, wherein the first camera and second camera remains stationary as the marker is moved from the first location to the second location.

2. The method of claim 1, wherein the marker is fixedly coupled to an object that includes a calibration pattern, wherein the object is at the first location captured in the first image and the second image, wherein the method further comprises determining, using a tracking device, a third location at which the marker is fixedly coupled to the calibration object, wherein determining the first pose of the first camera comprises:
determining a third pose of the first camera with respect to the calibration pattern while the object is at the first location; and
determining a fourth pose of the tracking device with respect to the calibration pattern using the third location of the marker.

3. The method of claim 2, wherein determining the fourth pose of the tracking device comprises:
determining a fifth pose of the tracking device with respect to the marker according to the third location of the marker;
retrieving a sixth pose of the marker with respect to the calibration pattern; and
adjusting the fifth pose according to the sixth pose.

4. The method of claim 2, wherein the marker and the calibration pattern is one integrated unit.

5. The method of claim 2, wherein the tracking device is an infrared (IR) sensor, and the marker is an IR tag.

6. The method of claim 2, wherein the tracking device is a camera, and the marker is a visible pattern.

7. A method performed by a surgical system that includes a first camera and a second camera that are located within an operating room, the method comprising:
receiving a first image captured by the first camera, the first image representing a first field of view (FOV) of the first camera including an object at a first location in the operating room;
determining a first pose of the first camera based on the first image and a tracking marker within the operating room;
receiving a second image captured by the second camera, the second image representing a second FOV of the second camera that does not overlap with the first FOV and having the object at a second location in the operating room;
determining a second pose of the second camera based on the second image and the tracking marker; and
determining a relative spatial transformation between the first camera and the second camera based on the first pose and the second pose.

8. The method of claim 7, wherein the object comprises a tracking device, wherein the method further comprises:
detecting, using the tracking device, the tracking marker while the object is at the first location, wherein the first pose is determined based on the detection of the tracking marker while the object is at the first location; and
detecting, using the tracking device, the tracking marker while the object is at the second location, wherein the second pose is determined based on the detection of the tracking marker while the object is at the second location.

9. The method of claim 8 further comprising tracking, using the tracking device, movement of the object from the first location to the second location, wherein the second pose is determined based on the tracked movement of the object.

10. The method of claim 8, wherein the tracking device and the object are one integrated unit.

11. The method of claim 8,
wherein the method further comprises determining a third pose of the tracking marker with respect to the object based on the detection of the tracking marker while the object is at the first location; and
determining a fourth pose of the first camera with respect to the object based on the first image, wherein the first pose of the first camera is based on the third pose and the fourth pose.

12. The method of claim 11, wherein determining the third pose of the tracking marker comprises:
determining a fifth pose of the tracking marker with respect to the tracking device based on the detection of the tracking marker;
receiving a sixth pose of the tracking device with respect to the object; and
adjusting the fifth pose according to the sixth pose.

13. The method of claim 8, wherein the tracking device is a tracking camera, wherein detecting the tracking marker while the object is at the first location comprises receiving a third image captured by the tracking camera, the third image representing a third FOV of the tracking camera and including the tracking marker.

14. The method of claim 8, wherein the tracking marker is an infrared (IR) tag, and the tracking device is an IR sensor, or the tracking marker is a radio frequency (RF) tag and the tracking device is a RF sensor.

15. The method of claim 7, wherein the object comprises a calibration pattern that is moved from the first location to the second location by a user or an autonomous robot within the operating room, while the first camera and the second camera remain in place.
